(19) United States
(12) Patent Application Publication
(10) Pub. No.: US 2023/0203621 A1
(43) Pub. Date: Jun. 29, 2023

(54) METHOD FOR FLEXIBLE MANUFACTURING OF INTERMETALLIC COMPOUNDS AND DEVICE FOR MAKING THEREOF

(71) Applicant: ALOTEK LTD, Sofia, Krasna polyana (BG)

(72) Inventors: TKACHUK Ivan, Sofia, Krasna polyana (BG); Volodymyr KOZYRSKYI, Sofia, Krasna polyana (BG)

(21) Appl. No.: 17/996,621

(22) PCT Filed: Apr. 21, 2020
(86) PCT No.: PCT/BG2020/000017

§ 371 Date: Oct. 20, 2022

(51) Int. Cl. C22C 1/02; C22C 9/00; G05B 19/042; G01N 1/44; G01N 33/20

(52) U.S. Cl. CPC C22C 1/02; C22C 9/00; G05B 19/042; G01N 1/44; G01N 33/20; G05B 2219/25257

(57) ABSTRACT

The invention relates to a method and apparatus for the flexible manufacture of intermetallic compounds, including those with shape memory effect. The method and the device can find mass application in the industrial production of modern functional and innovative products based on intermetallic compounds with predetermined physicomechanical parameters and properties. The method includes the steps of taking an intermediate sample of the meld, measuring the actual physico-mechanical properties and material characteristics of the sample and tuning the composition and/or the operating mode parameters of the melting furnace. The device includes measuring module (I) and module (II) for displaying and storing information.

### FIELD OF THE INVENTION

The invention relates to a method and apparatus for the flexible manufacture of intermetallic compounds, including those with shape memory effect. In particular, the invention relates to a method and apparatus for the flexible manufacture of intermetallic compounds using melting furnace, including manufacture via crucible or batch type induction furnaces working at atmospheric environment. The method and the device can find mass application in the industrial production of modern functional and innovative products based on intermetallic compounds with predetermined physico-mechanical parameters and properties.

Areas of application are numerous and varied, with examples of applications in the production of electricity, oil and gas, in the metallurgical, coal, chemical, food and other industries to create smart devices based on new functional materials. Materials based on the direct conversion of thermal energy, including low temperature, directly into mechanical operation, the so-called materials with shape memory effect, are particularly promising.

### BACKGROUND OF THE INVENTION

Metals, when melted, can react chemically with each other to form intermetallic compounds. Compounds between metals can be of different composition. Many intermetallic two-, three-component compounds based on various metals are known, such as Ti, Ni, Al, Cu, Au, Li, Na and others. They have various useful properties and improved mechanical, thermal, electrical, optical, magnetic, and other characteristics that make these materials increasingly sought after and applicable in the home and industry. The practical applications of intermetallic compounds are generally wide and varied. They are very strong construction materials, semiconductors, superconductors, materials for the preparation of permanent magnets and more. Intermetallic compounds are an important component of high temperature resistant alloys, typographic alloys, etc. Their application will increase in the future, and their widespread use will depend mainly on the provision of cheap and reliable technologies for their production. Their widespread application can be implemented, for example, in the manufacture of pipe connectors in the oil industry, retractable cell phone antenna, splints and brackets for orthodontic tooth correction, in the manufacture of toys and fun items, flexible eyeglass frames, flexible window frames, in the mass production of actuators, sensors, heat engines, lifting devices and more.

Some of the promising intermetallic compounds have the effect of restoring their form upon change in temperature or so-called shape memory effect. The basis of this effect is the process of phase transformation of the material, which takes place both in the deformation of the object and in the restoration of its shape. Such a change in shape produces a useful force that makes these intermetallic compounds effectively usable in a number of temperature sensitive devices with biomedical or technical applications. The chemical composition of the intermetallic compounds having shape memory effect provides the necessary transformation temperature and hysteresis width for the particular application. These important parameters can be controlled by varying the components involved of the composition by changing the ratios between them and/or by adding new elements to the melt.

Some of the well-known and commercialized materials are nickel-titanium materials, gold-cadmium or silver-cadmium materials, as well as materials based on copper, iron, and titanium. One of the most well-known and studied intermetals with shape memory effect is titanium nickelide, so called nitinol. So far, however, its application is limited mainly to the space industry and medicine. The main factor preventing the mass use of nitinol-containing materials containing Ti in other sectors of human activity is their high cost. It is due to both the expensive raw materials and the complexity of manufacturing technologies associated with significant difficulties due to the need for strict control over the composition and the extremely high chemical activity of titanium which requires special vacuum equipment.

Copper, manganese, and cobalt based intermetallic compounds are known, as well as manufacturing methods that utilize cheaper and more widespread starting components that do not always require the use of vacuum equipment. Some publications may be mentioned. For example, SU1624039A1 discloses a composition comprising copper, aluminum, manganese, cobalt and boron. The disclosure emphasizes the very accurate relationship between the starting components of the composition and the effect of changes in their ratios on the thermo-mechanical characteristics of the material with a high level of plastic deformability. However, accidental losses in the components, such as vapors, inaccurate and borderline dosing, etc., which can lead to other ratios that significantly alter the characteristics sought, are not taken into account. No means and steps are provided which, in the manufacturing process, can flexibly make the necessary adjustments to the proportions and modes to avoid scrapping the final compound.

SU1731859A1 discloses a method of thermal treatment of alloys from the Cu—Al—Mn system, comprising the steps of heating in the β-region and hardening, where before hardening annealing is carried out and the process being carried out under special heat treatment modes. This method of influencing the characteristics of the alloys obtained is important but insufficient as it has a very narrow scope of regulation for the characteristics of the alloys already obtained.

RU2327753C2 discloses a composition containing nickel, titanium, niobium and zirconium. Another publication CN110205538 is known, which discloses composition comprising nickel, titanium, niobium and aluminum. Another composition comprising titanium, nickel, copper and molybdenum is disclosed in KR20020004731(A). Here also the improvement of the characteristics of the alloys disclosed is achieved by preliminary very accurately determining the weight of the components before melting and introducing additional alloying elements such as niobium. They also lack means for determining the characteristics of the alloy during smelting, which significantly reduces the efficiency of this technology.

The technical solution described in RU2162900C1 discloses a method for the production of Ni—Ti system using a vacuum induction furnace, comprising the steps of preparing a feedstock mixture by accurately weighing the starting components of the charge, pre-lining the wall and bottom of the high-strength graphite crucible with nickel plates, placing the remaining components mixture in the crucible, melting in a vacuum induction furnace, retaining the alloy and pouring the alloy under vacuum in steel, cast iron or graphite casting molds. This method is not suitable for the mass production of many useful products based on intermetallic compounds with shape memory effect, for example Cu-based intermetallic compounds, as it relates to the production of an expensive titanium-based product which is made by expensive technology using vacuum induction furnaces. It requires very accurate measurement and dosing of the starting components.

It is known from US2018179620 (A1) a method of manufacturing alloys with shape memory effect comprising the steps of mixing nickel and at least one metalloid of the group containing germanium, antimony, zinc, gallium, lead, indium, bismuth and the rest of titanium, wherein the melt is heated in the range of 700 to 1300° C. for 50 to 200 hours. Aging of the alloy can be performed, as well as aluminum addition. This method is also not suitable for the mass production of intermetallic compound-based-products with shape memory effect, since an expensive titanium-containing product is used in vacuum furnaces. It requires very accurate measurement and dosing of the starting components. The method of producing an iron-based alloy with the effect of shape memory disclosed in JP2004115864 also requires very accurate measurement and dosing of the starting components, without providing flexible control of the production process.

Variety of methods of control are known in the metal production, which make the manufacturing process more reliable and the products obtained have a closer resemblance to the required properties.

It is known from US2008302503A1 a method for adaptively controlling the production of a metal alloy in a metallurgical furnace by preliminary calculating the amount of alloying components and the amount of initial and added base component. This known method includes the steps of: determining the amount of melt in the furnace and the amount of base component; calculation of expected physical and mechanical properties, such as melt strength and shrinkage index, using thermochemical analyzes and mathematical calculation models; computer comparison of the amount of forming agents in the starting composition to be introduced into the melt in the furnace and/or pot in order to provide the required physico-mechanical properties with the actual starting quantities of the components; continuously optimally determining the quantities to be added and adding the calculated amounts to the melt. This method has the advantage that the process is carried out in a single step and thereby optimally adjusts the melt composition within the predetermined process constraints. The method is suitable for application for small deviations from the initially set values of the embedded components. The disclosed method is not suitable for use in the process of mass production of intermetallic compounds, especially in smaller quantities, because it is not flexible and mainly depends on the very accurate initial determination of the amount of components used and their arrangement of layers in the furnace. The subsequent adjustment of the quantities should also be very precise, which greatly increases the cost of the process. Here, the qualities and properties of the cast specimen are judged indirectly by calculations and diagrams, not by direct verification.

The majority of the known technologies do not take into account all the factors. The achievement of the real quality characteristics and parameters of the obtained intermetallic compounds is determined only after the technological process is complete and the melt is poured into the molds. It is imperative that multiple attempts be made to obtain the melt of intermetallic compounds with the required characteristics and parameters. All this requires additional energy costs, labor costs, does not ensure efficient utilization of production capacities and ultimately leads to an increase in the cost of the obtained intermetallic compound product, thus reducing the competitiveness of these materials and limiting their use.

Known technologies for improving the performance of the resulting intermetallic compounds are forced to use different approaches, but they do not solve the general problem, but reveal only techniques that affect only some of the properties of the end product.

### SUMMARY OF THE INVENTION

It is an object of the present invention to provide a technology for the production of functional products based on intermetallic compounds in which the melting process is carried out in one step and through which functional compositions having the desired quality characteristics are achieved.

The object of the invention is achieved by a method for the flexible manufacturing of intermetallic compounds, comprising the steps of: inserting into a melting furnace casting starting components with predefined quantities and ratios based on predefined physical parameters and physico-mechanical characteristics for the finished product; melting of starting components under predetermined operating modes of the melting furnace; mixing and solidifying the melt to obtain a finished product of intermetallic compound, where prior to solidifying the melt to the finished product at least one time is taken at least once sample of the melt, analyzing the sample, and if necessary, adding further quantities and/or components under further mixing. Here flexible manufacturing means the ability of the method to easily adapt the entire production system by feedback from measurements of physico-mechanical parameters and characteristics of intermediate samples in order to meet future requirements and changes that will inevitably occur. According to the invention this is accomplished by the steps of: after each intermediate sample is taken, the step of solidifying the sample is carried out, and the analysis is carried out on the solidified sample by measuring the actual physico-mechanical properties and characteristics of the sample material, and, if necessary, adjusting the melt composition and/or parameters of the mode of the sample operation of the melting furnace.

The advantage of the method according to the invention lies in the immediate and direct measurement of the characteristics and properties of the finished product and the demonstration of the presence of the desired end effect when using an intermetallic compound. It is well known that the manufacturing of intermetallic compounds, including those with shape memory effect, as well as by melting the starting components in an open furnace under atmospheric pressure, is a substantially complex process. The properties and characteristics of the materials obtained are determined by many factors, including the exact quantity and quality of the components of the chemical composition of the melt. It is known that changes may occur during the process, for example due to inaccurate dosing or use of lower quality starting materials, loss of vapors, including inaccurate tuning of melting furnace modes. All this reflects the melt and the finished product can easily obtain other qualities and characteristics.

Therefore, it is very important to take timely steps to compensate for possible errors in determining the weight of the starting components or in determining the degree of purity of the prepared mixture. It is also necessary to be able to compensate for the inaccurately defined and set modes of the furnace or their flexible change in the melting process. These are factors that significantly affect the expected physico-mechanical including thermomechanical characteristics and parameters of the finished intermetallic compound. The technological process modes also have an effect, because as is known, irreversible changes in the melt can occur during the smelting process and this can lead to deviations in the characteristics and parameters of the obtained intermetallic compounds, including those with shape memory effect.

The proposed method directly controls exactly the desired properties and characteristics of the material and allows more than one time to flexibly compensate for inaccuracies and/or introduce other modes of operation of the furnace and/or make desired changes in the composition of the melt, providing the initially sought or newly set physico-mechanical properties and characteristics of the finished product. The method provides producing of a finished product with programmable properties during one smelting process and one loading of the melting furnace. The method provides effective accuracy in the preparation of the chemical composition of the starting components embedded in the melting furnace. It also provides the possibility of periodic monitoring of the functional parameters of intermediate samples corresponding to the finished products, which functional parameters depend on the ratio and type of the components of the melt. The method allows increasing the degree of automation in mass production. As a result, functional materials with the required characteristics determined by the production task are obtained without the melting process being repeated several times. The method guarantees a strong reduction or elimination of irreparable scrap from receiving irreversible changes in the chemical composition of the finished product. Thus, the method becomes effective and applicable both in small quantities and in large series. It can be used with success both in the single and mass production of modern functional and innovative products based on intermetallic compounds, including those with shape memory effect. The method makes it possible to increase the efficiency of technologies for the production of a wide range of intermetallic compounds with desirable and sought after properties and to expand the possibilities for their use in various fields of human activity.

In one embodiment of the method, the melting furnace is an open furnace operating in an atmospheric pressure. This avoids the use of special and expensive equipment where components whose production is not affected by atmospheric air and pressure are used.

In another embodiment of the method, the intermetallic compound is an intermetallic compound with shape memory effect, and the measured and/or preset physico-mechanical properties and characteristics of the intermediate solidified sample and/or of the finished product are thermomechanical properties and characteristics. Preferably, the intermetallic compound with shape memory effect is a Cu-based binary Cu—X or multi-element Cu—X—Y compound, where Y and/or X are selected from the elements of group II-VI of the periodic table. In this way the production is getting significantly cheaper and the possibilities for mass introduction of products manufactured by intermetallic compounds into the household and industry are greatly expanded.

In yet another embodiment of the invention, at least the amount and type of corrected and/or starting components of the melt, the corrected and/or initial modes of operation of the melting furnace, as well as the corresponding measured physico-mechanical properties and characteristics of the solidified sample material, are recorded in computer memory and form a working database. This provides the opportunity for complete process automation through easier selection of process data, providing the desired characteristics of the finished products.

It is also an object of the present invention to provide a device for analyzing intermediate solidified samples of intermetallic compounds useful in the implementation of the above described method for the flexible production of intermetallic compounds. The device according to the invention comprises at least one measuring module connected to a module for displaying and storing information. The measuring module(s) contain instruments capable of measuring the physico-mechanical properties and characteristics of the sample, with at least one of the measuring modules containing instruments for measuring the thermomechanical properties and characteristics of the sample. The information display and storage module comprises a controller connected to each of the measurement modules, which controller is capable of processing and storing data from the measurement of the physico-mechanical properties and characteristics of each intermediate solidified sample of intermetalling compound, as well as a display for controlling the measurement process. The information display and storage module also contains a memory and display to control the measurement of the intermediate solidified samples.

In one embodiment of the apparatus, instruments for measuring thermomechanical properties and sample characteristics include at least one strain gauge or tensometer and at least one of a pyrometer or a dilatometer, as well as a heater for changing the temperature of the measured intermediate sample to the required phase conversion temperature. This ensures that the device is operated when used in flexible manufacturing methods for intermetallic compounds, including those with shape memory effect.

In another embodiment of the apparatus, at least the information display and storage module is placed in a portable hand carrying container and the controller is a microcontroller capable of communicating with an external computer system. This embodiment makes it possible to use cheaper and versatile elements with simplified functions in the device itself, allowing the memory and complex computing processes to be able to run on an external computer. This helps to significantly reduce the price of the device as well as to make it a portable hand carrying device.

In another embodiment of the device according to the invention, the controller is a programmable controller capable of comparing measured data with predetermined values of physico-mechanical, including thermomechanical properties and material characteristics of the final intermetallic compound, having the ability to calculate the amount of individual components of the intermetallic compound composition to provide the physico-mechanical, including thermomechanical properties and material characteristics of the finished intermetallic compound product. This embodiment provides a device with more features, which is cost-effective for larger industries. Preferably, the controller is capable of signaling to executive devices or actuators of the entire casting system containing the casting furnace, as well as being able to manage databases containing values of the physico-mechanical, including thermomechanical properties and material characteristics of the final intermetallic compound, values of the quantities of individual components of the composition of the intermetallic compound and in some cases also containing values of operating modes of the casting furnace. Thus, the device according to the invention is designed as a stand-alone intelligent device for automatically controlling the process of manufacturing intermetallic compounds products.

### BRIEF DESCRIPTION OF THE DRAWINGS

FIG. 1 shows a functional diagram of the stages and steps of a method for manufacturing an intermetallic compound according to the invention.

FIG. 2 shows a functional block diagram of a device for analyzing intermediate samples of intermetallic compounds.

FIGS. 3 and 4 show thermomechanical characteristics of the intermetallic compound analyzed respectively at the final and intermediate measurements.

FIG. 5 shows the appearance of a portable hand carrying device for the analysis of intermediate samples of intermetallic compounds.

### DETAILED DESCRIPTION OF THE INVENTION

The present invention is demonstrated by the accompanying drawings, which shows one preferred embodiment of the method and apparatus more applicable to wide use.

The exemplary functional diagram shown in FIG. 1 illustrates the stages and steps of an exemplary method for manufacturing an intermetallic compound with shape memory effect having preset characteristics. Reference 1 indicates the stage of preparation of the starting components for the melt, comprising preparatory steps for determining the type and calculation of the amount of starting components in accordance with the required thermodynamic characteristics of the finished product and subsequent dosage by weight of the specified starting components in accordance with the requirements of the chemical composition. It is carried out in the ways known in the art of metal casting industry. In this case, this exemplary method is intended for the semi-automated production of a Cu-based binary Cu—Al or multi-element Cu—Zn—Al, Cu—Al—Mn, Cu—Ni—Al or Cu—Al—Zn intermetallic compound, whereby it is automated the process of analyzing the characteristics and properties of the material.

The method according to the invention is especially effective and suitable for use also in the manufacture of other intermetallic compounds as well as those for which no effect of shape memory is observed. These include intermetallic compounds whose components do not oxidize and can also be produced in open furnaces under atmospheric pressure. Such may be intermetallic compounds, for example Cu—Sn, Cu₆Sn₅, Li₂CuSn, LixCu₆Sn₅, Cu₂Sb.

The method can also be adapted and applied for vacuum or working with other gas environment induction furnaces used in the production of responsible functional products of expensive intermetallic compounds of the type Ti-based or based on other hard-melting and oxidizing metals. Such are compounds of the type Ni—Ti—Nb—Zr, Ni—Ti—Nb—Al, Ti—Ni—Cu—Mo, which have particularly responsible and special applications. Here, the process according to the invention can make the manufacturing process more efficient and reduce the risk of scrapping due to unfulfilled end parameters and characteristics of the finished product.

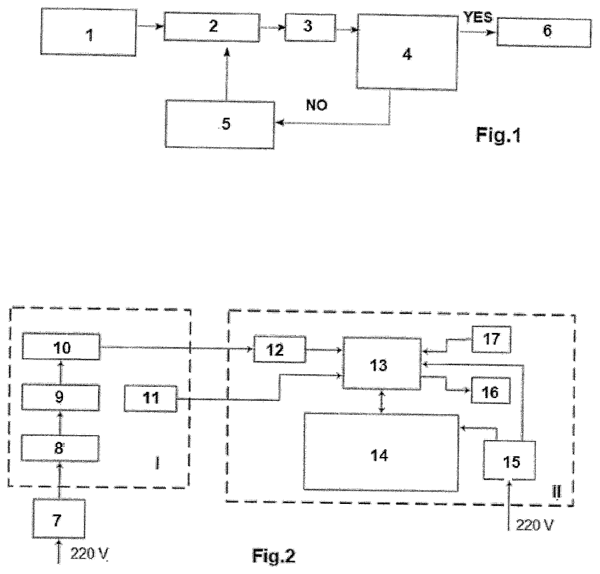

Stage 2 of the illustrated example provides loading the starting components into the crucible of an induction furnace with air-gas medium at atmospheric pressure and subsequently melting them according to the parameters of the operating mode of the melting furnace. Stage 3 provides the steps of taking an intermediate melt sample and solidifying the sample, which is carried out in standard and well-known ways of metal casting, as for example cooling at room temperature. During Stage 4 of the described example, steps are taken to determine and analyze the thermomechanical characteristics and parameters of the solidified intermediate sample by means of a specialized device (FIG. 2) for the analysis of a solid sample of an intermetallic compound obtained by the method of the invention. In the present example during Stage 4 a comparison of the measurements of thermomechanical characteristics and parameters of the solidified sample with the required thermodynamic characteristics of the finished product is also made. If the results of the compared measurements do not coincide and are outside the tolerance, Stage 5 is performed, in which steps of analyzing the cause and correcting the content of the individual components of the alloy and, if necessary, the modes of operation of the furnace are made. The time required to perform the analyses described does not exceed 0.5% of the total time of the complete process for the preparation of intermetallic compounds, incl. with shape memory effect.

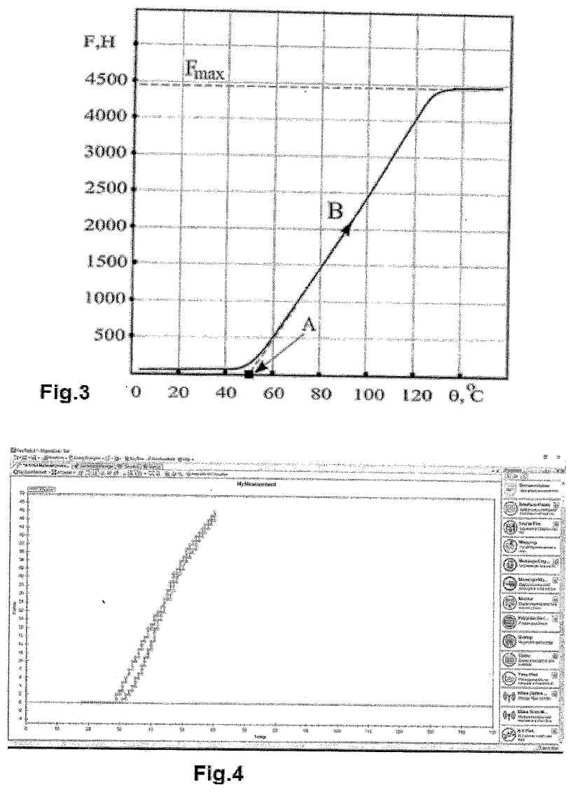

The steps of Stage 3, Stage 4, and Stage 5 are repeated until the required thermodynamic characteristics of the finished product are achieved (FIG. 3).

It is clear that the method is also suitable for manufacturing of intermetallic compounds by measuring of other physico-mechanical characteristics of intermediate solidified samples such as strength, stability, hardness, elasticity, plasticity, electrical conductivity and superconductivity, crystalline structure and other known and sought-after characteristics of intermetallic compounds, which measurements are made by using known measuring means.

After equalization of the measured characteristics of the sample and the required characteristics of the finished product, the method proceeds to Stage 6, where the melting process in the melting furnace is terminated and the melt casting is performed. Casting molds can be graphite or other type and should provide casting of the required shape and size, for example in the form of bars, tiles or other shapes.

The exemplary embodiment described illustrates the effectiveness of the proposed method. It is clear that the process is flexible and can very quickly be reconfigured as it progresses. Effective utilization of essential resources for the manufacturing of intermetallic compounds, including with the effect of shape memory, is ensured, as well as a significant reduction in production costs is achieved. The method increases its competitiveness and allows for a large extension of the use of such materials. The implementation of the proposed method can reduce energy consumption and increase the efficiency of using technological equipment and human resources by two or more times.

The implementation of the proposed technology is ensured by the use of a process-specific device for the analysis of solidified intermediate melt samples of an intermetallic compound. By using the device directly during the process, an express analysis of the thermomechanical and other physico-mechanical characteristics and parameters of the melt samples is carried out.

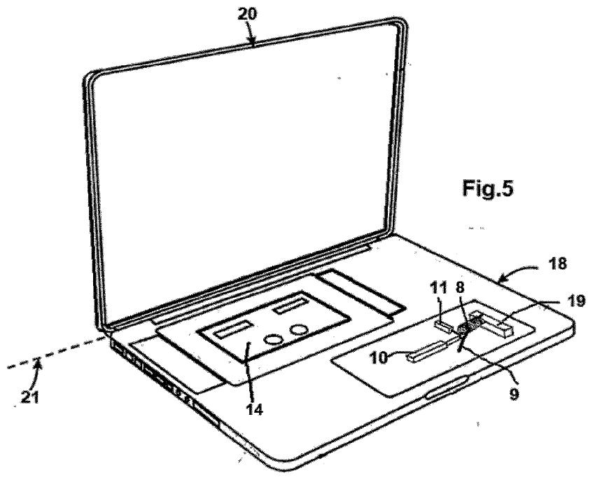

FIG. 2 shows one example of a functional block diagram of an apparatus for analyzing intermediate samples of intermetallic compounds according to the invention, and FIG. 5 shows an exemplary appearance of the device. The device is designed at modular principle and includes at least one measuring Module I, in this case a single module, and Module II for displaying and storing information. The measurement module I shown in FIG. 2 contains a power unit 7 and heater 8. The power unit 7 is connected at its input to a ˜220 V power supply network and at its output is connected to heater 8. A sample 9 of the cooled and solidified melt of the intermetallic compound, in the case with shape memory effect, is stationary fixed and located near the heater 8 so that it can be heated to the temperature required for phase conversion. The intermetallic compound in this case is a Cu-based, for example binary Cu—Al or multi-element Cu—Zn—Al, Cu—Al—Mn, Cu—Ni—Al or Cu—Al—Zn compound. In this example the measurement Module I also includes at least one strain gauge sensor or tensometer 10 connected to the sample 9 and at least one pyrometer or dilatometer 11. Pyrometer 11 is located adjacent to sample 9. In this example it is a non-contact temperature sensor of type MLX90614ESF-DCI. In the measurement Module I, the sample 9 is heated to a phase conversion temperature and the values of the temperature and the generated force from the sample 9 are measured. In this case, the strain gauge sensor 10 is a micro load device of type CZL635 and is designed to measure compressive forces. It works on the principle of changing the resistance of strain resistors.

The measurement modules may be separated and may include other known measuring means and instruments for measuring strength, stability, hardness, elasticity, plasticity, electrical conductivity and superconductivity, crystalline structure, magnetic properties and other known and sought after characteristics of intermetallic compounds.

The Module II for information display and data storage comprises a controller based on a microcontroller 13. In the exemplary embodiment the microcontroller 13 is of the ATmega2560 type. Module II further comprises a display 14 connected to the microcontroller 13. The display 14 in this case is touchscreen selected of type 7 “Nextion HMI LCD Touch Display. Pyrometer 11 is connected to the microcontroller 13 with the ability to transmit data from the corresponding measurement. An analog-to-digital converter 12, in the case of the HX-711 type, connects the strain gauge 10 to the microcontroller 13. Module II also contains a power supply unit 15 connected at its input to the ˜220 V power supply network and to the display 14 at its output. In addition, a memory 16, in this case type MicroSD, for data storage and a sensor 17 for measuring environment indications are connected to the microcontroller 13.

In this case, the information received in the course of the analysis from the microcontroller 13 is stored in the memory 16, and for further processing and use is submitted to a computer (not shown in the drawings), for example a personal computer.

In this case, the device for analyzing intermediate samples of intermetallic compounds is designed as a portable hand carrying device whose appearance, shown in FIG. 5, is similar to that of a laptop computer. The portable device comprises a body 18, inside which are housed the elements of Module II (not shown in the drawing)—the microcontroller 13, the analog-to-digital converter 12, the power supply unit 15, the memory 16 and the sensor 17 for measuring environmental indications. On the front panel of the body 18 in the example shown are mounted elements of Module I, as well as the display 14. The display 14 is a sensor type with the ability to set the START and STOP commands, as well as to display the measurement data obtained from the sensors—strain gauge 10 and pyrometer 11. The display 14 in other cases (not shown) may shows the measured data and/or comparison with predetermined values of the physico-mechanical, including thermomechanical properties. A holder 19 is shown in which the sample 9 is fastened. The heater 8, the pyrometer 11 and the tensometer 10 are appropriately mounted on the front panel of the body 18. The sample 9 in this case is in the form of a cylindrical wire which has been pre-deformed. It is appropriate, without limitation, for sample 9 to be 0.5 to 3 mm in diameter and 50 to 80 mm in length. The connection between the PC and the described device for analyzing intermediate samples of intermetallic compounds according to the invention is via a serial interface—USB cable—not shown. The device is provided with a cover 20 connected to the body 18 via a hinge 21. After turning on the power 7, the heater 8 heats the sample 9 to the phase conversion temperature measured with the pyrometer 11, the sample regains his upright position and the strain gauge sensor or tensometer 10 measures the force obtained.

For intermetallic compounds, it is known that the shape memory effect is characteristic of thermoelastic martensite, and the main parameters that determine the use of the compounds as functional materials are the temperature of the onset of the martensitic transformation, the temperature range of the memory effect, the amount of back deformation and the actual width of the temperature hysteresis.

FIGS. 3 and 4 show graphs of the dependence between thermomechanical properties of the intermetallic compound with a shape memory effect and the heating temperature obtained in the final and intermediate measurements, respectively. In FIG. 3, point A shows the temperature at which the restoration of the form of sample 9 begins, and reference B indicates the curve of thermomechanical characteristics of the sample during heating.

The above characteristics and advantages of the proposed method can be illustrated by the following examples. In all three examples, the above described device for analyzing intermediate samples of the invention was used.

### Example 1. A Process for Preparing an Intermetallic Compound with a Shape Memory Effect Having a Predetermined Temperature of 50+/−2° C. at the Beginning of Form Recovery

Pre-selected and weighted starting chemical components of the melt, e.g. Cu—Al—Mg, are placed in an open induction furnace at atmospheric pressure, preferably in a graphite crucible. The melting process is carried out in accordance with the functional scheme of the method of FIG. 1. During the melting process, three express analyzes of thermomechanical characteristics and melt parameters were performed, using rod-shaped solidified intermediate melt samples 1.5 mm in diameter and 80 mm in length. The temperature at the beginning of the form recovery process is recorded. After the analysis of each of the first two samples, the chemical composition of the melt was adjusted and tuned. After second tuning the third sample the target parameters of the finished product was achieved and the casting process was terminated.

The finished product is a 1 kg ingot with thermomechanical parameters corresponding to the set ones, with the achieved temperature at the beginning of the restoration of the form being 51° C., which is within the normal range. The error in determining the temperature at the beginning of the sample form recovery in the intermediate control process does not exceed 1%. The results of measurements and analyzes are shown in Table 1 below.

TABLE 1

| | | Actually measured |
|---|---|---|
| | Set temperature | temperature at |
| | at the beginning of | the beginning of |
| Sample number | form recovery, ° C. | form recovery, ° C. |
| 1 (starting components) | 50 +/− 2 | 64 |
| 2 (after first tuning) | 50 +/2 | 56 |
| 3 (after second tuning) | 50 +/2 | 51 |

### Example 2. A Process for Preparing an Intermetallic Compound with Shape Memory Effect Having a Predetermined Temperature of 70+/−2° C. at the Beginning of Form Recovery

The same conditions as Example 1 were performed in the same manner. A final sample with a target temperature at the beginning of form recovery of 69.5° C. was obtained. The results are shown in Table 2

TABLE 2

| | | Actually measured |
|---|---|---|
| | Set temperature | temperature at |
| | at the beginning of | the beginning of |
| Sample number | form recovery, ° C. | form recovery, ° C. |
| 1 (starting components) | 70 +/− 2 | 61 |
| 2 (after first tuning) | 70 +/2 | 66 |
| 3 (after second tuning) | 70 +/2 | 69.5 |

### Example 3. A Process for Preparing an Intermetallic Compound with Shape Memory Effect Having a Predetermined Temperature of 90+/−2° C. at the Beginning of Form Recovery

The same conditions as Example 1 were carried out in the same manner. A final sample with a target temperature at the beginning of form recovery of 91° C. was obtained. The results are shown in Table 3.

TABLE 3

| | | Actually measured |
|---|---|---|
| | Set temperature | temperature at |
| | at the beginning of | the beginning of |
| Sample number | form recovery, ° C. | form recovery, ° C. |
| 1 (starting components) | 90 +/− 2 | 105 |
| 2 (after first tuning) | 90 +/2 | 82 |
| 3 (after second tuning) | 90 +/2 | 86 |
| 4 (after third tuning) | 90 +/2 | 91 |

The above examples show that the use of the method and device according to the invention makes it possible to reach the finished product of intermetallic compound from one melt by adjusting the properties of the melt during the process, both in case of exceeding the expected value of the required temperature and in the case of its lower value.

All finished intermetallic products have the necessary physico-mechanical, in the showing cases thermomechanical characteristics and parameters, including the required temperature at the beginning of form recovery of the finished product.

Although the description above contains many specifics, these should not be construed as limiting the scope of the invention but as merely providing illustrations of some of the presently preferred embodiments of this invention. Thus, the scope of this invention should be determined by the appended claims and their legal equivalents.

1. A method for the flexible manufacturing of intermetallic compounds, comprising the steps of:
inserting into a melting furnace starting components with predefined quantities and ratios based on predefined physical parameters and physico-mechanical characteristics for the finished product,
melting of the starting components under predetermined operating modes of the melting furnace, mixing and solidifying the melt to obtain a finished product of an intermetallic compound, wherein
i) prior to solidifying the melt to the finished product at least one time is taken at least one intermediate sample of the melt, where the size of at least one taken sample is smaller than the finished product, analyzing the at least one taken sample, and if necessary, adding further quantities and/or components under further mixing,
ii) after taking the sample (9), the step of solidifying the sample (9) is carried out;
iii) analyzing the solidified sample (9), including measuring the physico-mechanical properties and material characteristics of the sample (9); and
iv) if necessary, the operating mode parameters of the melting furnace are also corrected.

2. A method according to claim 1, wherein the melting furnace is a furnace operating at atmospheric pressure.

3. A method according to claim 1, wherein the intermetallic compound is an intermetallic compound with shape memory effect, and the measured and/or preset physico-mechanical properties and characteristics of the solidified sample (9) and/or of the finished product are thermomechanical properties and characteristics.

4. A method according to claim 3, wherein the intermetallic compound with shape memory effect is a Cu-based binary Cu—X or multi-element Cu—X—Y compound, where Y and/or X are selected from the elements of II-VI group of the periodic table.

5. A method according to claim 1, wherein at least the quantities and type of the corrected and/or starting components of the melt, the corrected and/or initial modes of operation of the melting furnace, as well as the corresponding measured physico-mechanical properties and characteristics of the solidified sample (9) are stored in memory (16) and form a working database.

6. A device suitable for analyzing of solidified samples of intermetallic compounds resulting from the method for the flexible manufacturing of intermetallic compounds of claim 1, wherein it includes:
i) at least one measuring module (I) comprising instruments for measuring physico-mechanical properties and characteristics of at least one solidified intermediate sample (9) taken from a melt of the melting furnace, the measuring instruments include instruments (10, 11) for measuring thermomechanical properties and characteristics of the solidified sample (9) of intermetallic compounds;
ii) a module (II) for displaying and storing information comprising a controller (13) for processing and memory (16) for storing data from the measurement of the physico-mechanical properties and characteristics of each solidified sample (9) of intermetallic compounds;
iii) the module (II) further comprising a display (14) to control the measurement process of the intermediate solidified samples (9).

7. A device according to claim 6, wherein the instruments for measuring thermomechanical properties and sample characteristics include at least one of a strain gauge or tensometer (10) and at least one of a pyrometer or dilatometer (11), as well as a heater (7) for changing the temperature of the measured sample (9) of intermetallic compounds to the required phase conversion temperature of the sample material.

8. A device according to claim 6, wherein at least the information display and storage module (II) is placed in a portable hand carry container (18) and the controller (13) is a microcontroller capable of communicating with an external computer system.

9. A device according to claim 6, wherein the controller (13) is a programmable controller capable of:
i) comparing the measured data with predetermined values of the physico-mechanical properties, including thermomechanical properties and material characteristics of the finished intermetallic compound product, and
ii) calculating the amount of the individual components of the composition of the intermetallic compound to provide the physico-mechanical properties, including thermomechanical properties and material characteristics of the finished intermetallic compound product.

10. A device according to claim 9, wherein the controller (12) is capable of signaling to executive devices or actuators of the casting system, and is capable of managing databases containing values of physico-mechanical properties and characteristics of the material of the final intermetallic compound, values of the quantities or amounts of the individual components of the composition of the intermetallic compound, and values of the modes of operation of the cast furnace.

11. An apparatus for analyzing samples of intermetallic compounds comprising:
i) a measuring module (I), comprising
(a) a heater (8) for heating at least one solidified intermediate sample (9) taken from a melt of a melting furnace to a predetermined temperature,
(b) instruments (10, 11) for measuring physico-mechanical properties and characteristics of the at least one solidified intermediate sample (9), said instruments comprising instruments for measuring thermomechanical properties and characteristics of the solidified intermediate sample (9);
ii) a module (II) for displaying and storing information and controlling the measurement process, comprising
(c) a controller (13) for processing data from the measurement of the physic-mechanical properties and characteristics of each solidified intermediate sample (9), said controller (12) connected to the measuring instruments;
(d) a memory (16) for storing data, and
(e) a display (14) for visualizing data, and for assisting in the control of the measurement process of the solidified intermediate samples (9).

12. An apparatus according to claim 11 further comprising an analog to digital converter (12) for converting analog data received from module (I) into digital format.

13. An apparatus according to claim 11 further comprising a sensor (17) for measuring environment indications and providing measured environment indications to the controller (13).

14. An apparatus according to claim 11, wherein module (I) and module (II) are provided in a portable housing (18), capable of being hand-carried.

15. An apparatus according to claim 11, wherein the instruments comprise at least one of a strain gauge sensor or tensometer (10) and at least one of a pyrometer or dilatometer (11).

16. An apparatus according to claim 11, further comprising a holder for stationary fixing at least one solidified intermediate sample (9) and located near the heater (8) so that the sample (9) can be heated to a temperature required for phase conversion of said intermetallic compounds.

17. An apparatus according to claim 11, further comprising a communications port for connecting to a processing computer and providing to said computer measured or stored data.

18. An apparatus according to claim 14, further comprising a protective cover (20).
